SEQUENCE LISTING (2 sequences)

SEQ ID NO: 1 - DNA, 45 bp, Artificial Sequence
  Synthetic- primer
gggtcgactaatacgactcctatagtaaaacagctctggggttgt

SEQ ID NO: 2 - DNA, 22 bp, Artificial Sequence
  Synthetic- primer
ccatttctcgggcactggagcg

(19) United States
(12) Patent Application Publication
(10) Pub. No.: US 2022/0387529 A1
(43) Pub. Date: Dec. 8, 2022

(54) TREATMENT FOR PRIMARY AND METASTATIC CANCER

(71) Applicant: Duke University, Durham, NC (US)

(72) Inventors: Darell BIGNER, Mebane, NC (US); Matthias GROMEIER, Durham, NC (US); Smita K. NAIR, Cary, NC (US)

(21) Appl. No.: 17/774,329

(22) PCT Filed: Nov. 4, 2020
(86) PCT No.: PCT/US2020/058812

§ 371 Date: May 4, 2022

Related U.S. Application Data
(60) Provisional application No. 62/963,642, filed on Jan. 21, 2020.
(60) Provisional application No. 62/930,010, filed on Nov. 4, 2019.

(51) Int. Cl. A61K 35/768; A61P 35/04; A61K 39/13

(52) U.S. Cl. CPC A61K 35/768; A61P 35/04; A61K 39/13; A61K 2121/00

(57) ABSTRACT

Provided is a method of treating a distal tumor in an individual by administering a chimeric poliovirus to a first tumor in an effective amount to induce an antitumor immune response effective to treat a distal tumor.

### CROSS-REFERENCE TO RELATED APPLICATIONS

This invention claims priority to U.S. Provisional Application No. 62/930,010 filed on Nov. 4, 2019 and U.S. Provisional Application No. 62/963,642 filed on Jan. 21, 2020, the contents of which are both incorporated by reference in their entireties.

### STATEMENT REGARDING FEDERALLY SPONSORED RESEARCH

N/A

### SEQUENCE LISTING

A Sequence Listing accompanies this application and is submitted as an ASCII text file of the sequence listing named “155554_00571_ ST25.txt” which is 676 bytes in size and was created on Nov. 4, 2020. The sequence listing is electronically submitted via EFS-Web with the application and is incorporated herein by reference in its entirety.

### TECHNICAL FIELD OF THE INVENTION

This invention is related to the area of anti-tumor therapy. In particular, it relates to oncolytic virus anti-tumor treatment including treatment of metastases or tumors distal to a treated tumor.

### BACKGROUND OF THE INVENTION

PVSRIPO is a recombinant oncolytic poliovirus (also referred to herein as “chimeric poliovirus”). It consists of the live attenuated type 1 (Sabin) PV vaccine containing a foreign internal ribosomal entry site (IRES) of human rhinovirus type 2 (HRV2)(FIG. 1). The IRES is a cis-acting genetic element located in the 5′ untranslated region of the poliovirus genome, mediating viral, m7G-cap-independent translation. The anti-tumor effects of PVSRIPO comprise direct, virus-mediated tumor cell killing; and infection of antigen presenting cells in induction of a potent host-mediated immune response directed against tumor. Thus, PVSRIPO takes advantage of its marked tropism for infection and killing of solid cancers mediated by natural ectopic over-expression of the human poliovirus receptor, CD155 (also known as Nectin-like molecule 5 (Necl-5)) on the surface of tumor cells, and infection and activation of antigen presenting cells (e.g., dendritic cells, macrophages) via natural expression of CD155 on their surface; thereby initiating a broad range of pro-inflammatory and immunogenic events that may recruit adaptive immune effector responses against the tumor. PVSRIPO is administered directly to a tumor and is not capable of spreading to tumors distant to the site of intratumoral administration. U.S. Pat. No. 10,398,743 describes use of PVSRIPO in treatment of primary tumor by intratumoral administration to the primary tumor.

Oncolytic viruses can vary greatly in their ability to effect an antitumor response, depending on viral properties (e.g., ability to infect and kill tumor cells, armed or unarmed, etc.) and the properties of the immune response induced (e.g., mechanism, potency, durability, and type). For example, oncolytic viruses can be armed or unarmed. Armed oncolytic viruses are viruses which code for human cytokines intended to potentiate an antitumor immune response. For example, talimogene laherparepvec (also known as T-Vec) is a genetically manipulated Herpes Simplex Virus 1 with an affixed granulocyte macrophage colony-stimulating factor (GM-CSF); and TILT-123 is an oncolytic adenovirus encoding human tumor necrosis factor alpha (TNF-α) and interleukin 2 (IL-2). PVSRIPO is considered an unarmed virus. In a comparison to unarmed viruses, armed viruses demonstrated an abscopal effect, whereas unarmed viruses did not (see, e.g., Havunen et al., 2018, Molecular Therapy: Oncolytics, 11:109-121). An abscopal effect is administration of an oncolytic virus to a first tumor (also referred to a “local tumor”), wherein the virus is not capable of spreading to tumor distant to the local tumor (also referred to as distal tumor or metastases); however, the administering the oncolytic virus to the first tumor induces a systemic immune response effective to treat distal tumor. It is estimated that metastases are responsible for about 90% of cancer deaths. Thus, there is a continuing need in the art to identify and develop anti-cancer treatments that provide one or more improved therapeutic benefits to humans, particularly for individuals with metastases or other distal tumor.

### SUMMARY OF THE INVENTION

Provided is a method of treating a distal tumor in an individual having a first tumor, optionally which expresses NECL5, and one or more tumors distal to the first tumor, the method comprising:

contacting tumor cells of the first tumor with a human a chimeric poliovirus comprising a Sabin type I strain of poliovirus with a human rhinovirus 2 (HRV2) internal ribosome entry site (IRES) in said poliovirus' 5′ untranslated region between said poliovirus' cloverleaf and said poliovirus' open reading frame, in forming a first tumor treated with the chimeric poliovirus, and wherein the one or more distal tumors are not contacted with the chimeric poliovirus;
wherein treatment of the first tumor in the individual results in a reduction of one or more tumor properties of one or more of the distal tumors comprising a reduction in one or more of rate of tumor growth or proliferation, tumor size, or tumor burden. The first tumor and distal tumor each comprise a solid tumor. For cancer in which multiple occurrences of tumor develop on the same organ (e.g., melanoma and the skin), a distal tumor may comprise a tumor several centimeters (e.g., on the same limb) or more (on a different limb) from a first tumor. The distal tumor may comprise a metastases of the first tumor; a tumor of the same tumor type (e.g., both the first tumor and distal tumor may be a melanoma); or a tumor type different than the first tumor but sharing one or more tumor antigens with the first tumor (e.g., first tumor may be a head and neck tumor, and the distal tumor may be a glioma). The method may further comprise a reduction in in one or more of rate of tumor growth or proliferation, tumor size, or tumor burden, in the first tumor as a result of treatment with the chimeric poliovirus. The method may further comprise administering a poliovirus vaccine booster (e.g., inactivated polio vaccine trivalent inactivated IPOL from Sanofi-Pasteur), or oral polio vaccine booster) between 6 months and 1 week prior to administering chimeric poliovirus to the first tumor. The method may further comprise administering therapeutically effective amount of an immune checkpoint inhibitor to the individual either prior to, concurrent with, or subsequent to, treatment of the first tumor with chimeric poliovirus. The method may further comprise multiple administrations of the chimeric poliovirus to the first tumor. The method may further comprise achieving a complete pathologic response in distal tumors.

Provided is a method of treating one or more distal tumors in an individual having a first tumor, which optionally expresses NECL5, the method comprising: contacting tumor cells of the first tumor with a chimeric poliovirus comprising a Sabin type I strain of poliovirus with a human rhinovirus 2 (HRV2) internal ribosome entry site (IRES) in said poliovirus' 5′ untranslated region between said poliovirus' cloverleaf and said poliovirus' open reading frame forming a first tumor treated with the chimeric poliovirus, and wherein the one or more distal tumors are not contacted with the chimeric poliovirus; monitoring one more distal tumors in the individual for a change in tumor properties following contacting tumor cells of the first tumor with the chimeric poliovirus;

wherein treatment of the first tumor in the individual results in a reduction of one or more tumor properties of one or more of the distal tumors, wherein the one or more tumor properties comprise a reduction in one or more of rate of tumor growth or proliferation, tumor size, or tumor burden. The first tumor and distal tumor may each comprise a solid tumor. For cancer in which multiple occurrences of tumor develop on the same organ (e.g., melanoma and the skin), a distal tumor may comprise a tumor several centimeters (e.g., on the same limb) or more (on a different limb) from a first tumor. The distal tumor may comprise a metastases of the first tumor; a tumor of the same tumor type (e.g., both the first tumor and distal tumor may be a melanoma); or a tumor type different than the first tumor but sharing one or more tumor antigens (“shared tumor antigen”) with the first tumor (e.g., first tumor may be a head and neck tumor, and the distal tumor may be a glioma). The method may further comprise a reduction in one or more of rate of tumor growth or proliferation, tumor size, or tumor burden, in the first tumor as a result of treatment with the chimeric poliovirus. The method may further comprise administering a poliovirus vaccine booster (e.g., inactivated polio vaccine trivalent inactivated IPOL from Sanofi-Pasteur), or oral polio vaccine booster) between 6 months and 1 week prior to administering chimeric poliovirus to the first tumor. The method may further comprise administering a therapeutically effective amount of an immune checkpoint inhibitor to the individual either prior to, concurrent with, or subsequent to, treatment of the first tumor with chimeric poliovirus. The method may further comprise multiple administrations of the chimeric poliovirus to the first tumor. The method may further comprise achieving a complete pathologic response in distal tumors.

Provided is a chimeric poliovirus comprising a Sabin type I strain of poliovirus with a human rhinovirus 2 (HRV2) internal ribosome entry site (IRES) in said poliovirus' 5′ untranslated region between said poliovirus' cloverleaf and said poliovirus' open reading frame, for use in treating a distal tumor in an individual having a first tumor which expresses NECL5 and one or more tumors distal to the first tumor, wherein the chimeric poliovirus is administered to the first tumor. Administration of the chimeric virus to the first tumor induces an antitumor immune response which results in treatment of distal tumor in the individual. The observed effect on distal tumor can be detected by monitoring distal tumor subsequent to treatment of the first tumor. The observed effect may comprise a complete pathologic response in distal tumors.

Treatment of the individual with any methods of the invention may result in a therapeutic benefit comprising one or more of: improved overall survival; improved disease-free survival; decreased likelihood of recurrence (in the primary organ and/or distant recurrence); decreased incidence of metastatic disease; an increased antitumor immune response; or an improvement in overall objective response rate using the appropriate response assessment criteria known to those skilled in the art and depending on the type of cancer treated (e.g., for lymphoma, see Cheson et al., 2014, J. Clin. Oncology32 (27):3059-3067; for solid nonlymphoid tumors, Response Evaluation Criteria In Solid Tumors (RECIST).

These and other aspects which will be apparent to those of skill in the art upon reading the specification provide the art with new therapeutic regimens for treating cancer.

### BRIEF DESCRIPTION OF THE DRAWINGS

FIG. 1 is a diagram depicting the genetic structure of oncolytic chimeric poliovirus, PVSRIPO.

FIG. 2A is photo representing three melanoma tumors on the neck of Patient A prior to treatment with PVSRIPO.

FIG. 2B is a photo representing three melanoma tumors on the neck of Patient A twenty one days following treatment of tumor 1 with PVSRIPO.

FIG. 2C is a photo representing three melanoma tumors on the neck of Patient A forty two days following treatment of tumor 1 and 21 days following treatment of tumor 2 with PVSRIPO.

FIG. 2D is a CT scan of tumor 1, before treatment with chimeric poliovirus, on the neck of Patient A. Tumor lesion is denoted by the circle.

FIG. 2E is a CT scan of the treated tumor 1 on the neck of Patient A, 63 days after treatment with chimeric poliovirus. The area representing the tumor treated is denoted by the circle.

FIG. 3A is photo representing melanoma tumors on a leg of Patient B prior to treatment with PVSRIPO.

FIG. 3B is a photo representing melanoma tumors on a leg of Patient B twenty one days following treatment of a first tumor with PVSRIPO.

FIG. 3C is a photo representing melanoma tumors on a leg of Patient B forty two days following treatment of a first tumor and 21 days following treatment of a second tumor with PVSRIPO.

FIG. 3D is a photo representing the area where melanoma tumors previously were observed on a leg of Patient B ninety days following treatment of a first tumor with PVSRIPO.

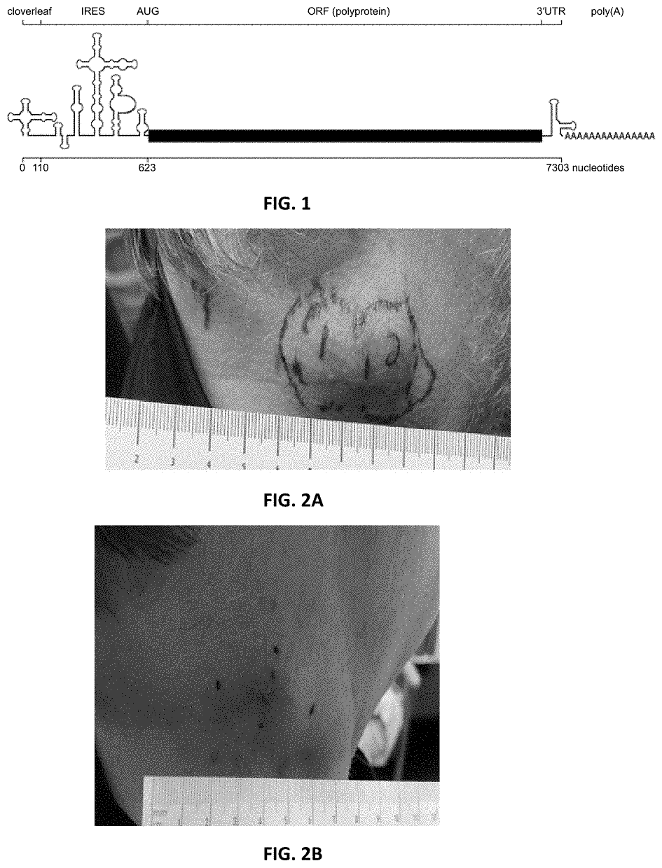

### DETAILED DESCRIPTION OF THE INVENTION

The present disclosure provides methods of treating one or more distal tumors in a subject. The method is based on the surprising and unexpected effect that locally treating a primary tumor with a chimeric poliovirus (i.e. PVSRIPO) resulted in an antitumor immune response effective to reduce one or more properties of distal tumor. The chimeric poliovirus is not administered to the one or more distal tumors, but a surprising reduction in one or more tumor properties of the distal tumors is seen. Suitably, the subject is a subject that has failed one or more standard of care treatments for the tumor type (e.g., checkpoint inhibitor therapy, etc.).

Provided is a method of treating a distal tumor in an individual having a first tumor, which optionally expresses NECL5, and one or more tumors distal to the first tumor, the method comprising:

contacting tumor cells of the first tumor with a chimeric poliovirus forming a first tumor treated with the chimeric poliovirus, and wherein the one or more distal tumors are not contacted with the chimeric poliovirus; optionally, monitoring one more distal tumors in the individual for a change in tumor properties following contacting tumor cells of the first tumor with the chimeric poliovirus; wherein treatment of the first tumor in the individual results in a reduction of one or more tumor properties of the distal tumors. The reduction in one or more properties in the distal tumor comprise a reduction in one or more of rate of tumor growth or proliferation, tumor size, or tumor burden. Unexpectedly, treatment of a first tumor locally with the chimeric poliovirus results in an antitumor immune response effective to reduce one or more properties of distal tumor. Optionally, the method may further comprise administering to the individual a maintenance therapy comprising the one or more immunotherapeutic agents.

The subject or patient is a human, preferably a human with cancer, suitably metastatic cancer with one or more distal tumors. In some embodiments, the subject is a subject with cancer that has failed one or more standard of care cancer therapies for the tumor type. For example, the patient has failed checkpoint inhibitor therapy and /or standard chemotherapy.

For example, as demonstrated in the Examples, subject has failed standard of care treatment for that tumor type (e.g., checkpoint inhibitors).

In the methods of the invention, any technique for contacting the first tumor with a chimeric poliovirus may be used. Contacting includes direct contact or direct administration of the chimeric poliovirus to the tumor. Direct administration does not rely on the blood vasculature to access the tumor. Suitable methods of direct contact are known in the art. For example, the preparation may be painted on the surface of the tumor, injected into the tumor (inter-tumoral administration), instilled or injected in or at the tumor site, infused into the tumor via a catheter, etc.

In some embodiments, the method may further comprise administering to the individual a maintenance therapy. In some embodiments, the maintenance therapy is one or more immunotherapeutic agents. Suitable immunotherapeutic agents are known in the art, and include, for example, immune checkpoint inhibitors, antibody therapies, and the like.

Immune checkpoint inhibitors which may be used according to the invention are any that disrupt the inhibitory interaction of cytotoxic T cells and tumor cells. These include but are not limited to anti-PD-1 antibody, anti-PD-L1 antibody, anti-CTLA4 antibody, anti- LAG-3 antibody, and/or anti-TIM-3 antibody. Commercially available and approved checkpoint inhibitors in the U.S. include Atezolizumab (PD-L1), ipimilumab (CTLA-4), pembrolizumab (PD-1), nivolumab (PD-1), avelumab (PD-L1), durvalumab (PD-L1), cemiplimab (PD-1), and tislelizumab PD-1). The inhibitor need not be an antibody, but can be a small molecule or other polymer. Structures and potencies of several small molecule inhibitors of PD-L1 have been published (see, e.g., Guzik et al. Molecules 2019, 24:2071). If the inhibitor is an antibody it can be a polyclonal, monoclonal, fragment, single chain, or other antibody variant construct. Inhibitors may target any immune checkpoint known in the art, including but not limited to, CTLA-4, PDL1, PDL2, PD1, B7-H3, B7-H4, BTLA, HVEM, TIM3, GALS, LAG3, VISTA, KIR, 2B4, CD160, CGEN-15049, CHK 1, CHK2, A2aR, and the B-7 family of ligands. Combinations of inhibitors for a single target immune checkpoint or different inhibitors for different immune checkpoints may be used. Additionally, colony stimulating factor 1 receptor (CSF-1R) blockade may be used in combination or as an alternative to immune checkpoint inhibitor(s), to ensure generation of potent and sustained immunity that effectively eliminates distant metastases and recurrent tumors. Antibodies specific for CSF-1R or drugs that inhibit or blockade CSF-1R may be used for this purpose, including but not limited to emactuzumab (RG7155 and RO5509554, Celleron Therapeutics, see e.g., Ries CH, et al. (June 2014). “Targeting tumor-associated macrophages with anti-CSF-1R antibody reveals a strategy for cancer therapy”. Cancer Cell. 2S (6): 846-59; who.int/medicines/publications/druginformation/innlists/PL111.pdf p. 232, incorporated by reference in their entirety) and AMG820 (see, e.g., WO2009026303, incorporated by reference).

Checkpoint inhibitors that comprise anti-PD1 antibodies or anti-PDL1-antibodies or fragments thereof are known to those skilled in the art, and include, but are not limited to, cemiplimab, nivolumab, pembrolizumab, MEDI0680 (AMP-514), spartalizumab, camrelizumab, sintilimab, toripalimab, dostarlimab, and AMP-224. Checkpoint inhibitors that comprise anti-PD-L1 antibodies known to those skilled in the art include, but are not limited to, atezolizumab, avelumab, durvalumab, and KN035. The antibody may comprise a monoclonal antibody (mAb), chimeric antibody, antibody fragment, single chain, or other antibody variant construct, as known to those skilled in the art. PD-1 inhibitors may include, but are not limited to, for example, PD-1 and PD-L1 antibodies or fragments thereof, including, nivolumab, an anti-PD-1 antibody, available from Bristol-Myers Squibb Co and described in U.S. Pat. Nos. 7,595,048, 8,728,474, 9,073,994, 9,067,999, 8,008,449 and 8,779,105; pembrolizumab, and anti-PD-1 antibody, available from Merck and Co and described in U.S. Pat. Nos. 8,952,136, 83,545,509, 8,900,587 and EP2,170,959; atezolizumab is an anti-PD-L1 available from Genentech, Inc. (Roche) and described in U.S. Pat. No. 8,217,149; avelumab (Bavencio, Pfizer, formulation described in PCT Publ. W02017097407), durvalumab (Imfinzi, Medimmune/AstraZeneca, W02011066389), cemiplimab (Libtayo, Regeneron Pharmaceuticals Inc., Sanofi, see, e.g., U.S. Pat. No. 9,938,345 and 9,987,500), spartalizumab (PDR001, Novartis), camrelizumab (AiRuiKa, Hengrui Medicine Co.), sintillimab (Tyvyt, Innovent Biologics/Eli Lilly), KN035 (Envafolimab, Tracon Pharmaceuticals, see, e.g., WO2017020801A1); tislelizumab available from BeiGene and described in U.S. Pat. No. 8,735,553; among others and the like. Other PD-1 and PD-L1 antibodies that are in development may also be used in the practice of the present invention, including, for example, PD-1 inhibitors including toripalimab (JS-001, Shanghai Junshi Biosciences), dostarlimab (GlaxoSmithKline), INCMGA00012 (Incyte, MarcoGenics), AMP-224 (AstraZeneca/MedImmune and GlaxoSmithKline), AMP-514 (AstraZeneca), and PD-L1 inhibitors including AUNP12 (Aurigene and Laboratoires), CA-170 (Aurigen/Curis), and BMS-986189 (Bristol-Myers Squibb), among others (the references citations regarding the antibodies noted above are incorporated by reference in their entirities with respect to the antibodies, their structure and sequences). Fragments of PD-1 or PD-L1 antibodies include those fragments of the antibodies that retain their function in binding PD-1 or PD-L1 as known in the art, for example, as described in AU2008266951 and Nigam et al. “Development of high affinity engineered antibody fragments targeting PD-L1 for immunoPED,” J Nucl Med May 1, 2018 vol. 59 no. supplement 1 1101, the contents of which are incorporated by reference in their entireties.

Optionally, the method of the invention may further comprise administration of a therapeutically effective amount of an immune checkpoint inhibitor 30 days or more prior to an individual being treated with the chimeric poliovirus. Optionally, following treatment with the chimeric poliovirus, the individual may undergo maintenance therapy beginning observance of a reduction in one or more tumor properties of distal tumor. Standard intervals for administration of an effective amount of immune checkpoint inhibitor in maintenance therapy is eight weeks, but the frequency may be adjusted (e.g., 2, 3, 4 ,or 6 weeks) based on tumor response and patient health as determined by a medical practitioner. A therapeutically effective amount of an immune checkpoint inhibitor may range from about 0.5 mg/kg of body weight to about 5 mg/kg of body weight; from about 1 mg/kg of body weight to about 5 mg/kg of body weight; from about 1 mg/kg of body weight to about 3 mg/kg of body weight; from about 500 mg to about 1500 mg, or lesser or greater amounts as determined by a medical practitioner. An immune checkpoint inhibitor may be administered by any appropriate means known in the art for the particular inhibitor. These include intravenous, oral, intraperitoneal, sublingual, intrathecal, intracavitary, intramuscularly, intratumorally, and subcutaneously. An immune checkpoint inhibitor may further comprise a pharmaceutically acceptable carrier.

In one aspect of the invention, a therapeutically effective amount of the chimeric poliovirus is an amount effective to cause a therapeutic benefit to an individual receiving the chimeric poliovirus. In one aspect, a therapeutic benefit comprises a reduction of one or more tumor properties of the distal tumors comprising a reduction in one or more of rate of tumor growth or proliferation, tumor size, or tumor burden. Such an effective amount may also vary according to characteristics of the individual, including health status, gender, size (e.g., body weight), age, cancer type, cancer stage, route of administration, tolerance to therapy, toxicity or side effects, and other factors that a skilled medical practitioner would take into account when establishing appropriate treatment dosing and regimen. For example, a therapeutically effective amount of an oncolytic chimeric poliovirus may range from about 1×10⁸ tissue culture infectious dose (TCID) to about 5×10⁵ TCID. The chimeric poliovirus may further comprise a pharmaceutically acceptable carrier.

Human tumor to be treated by a method of the invention includes solid tumors, and may comprise pediatric tumors or adult tumors. It is the unique ability of the chimeric poliovirus to infect and activate antigen presenting cells (for example, by inducing a sustained type I interferon-dominant proinflammatory stimulation of antigen-presenting cells) to induce a potent antitumor response (for example, immune cell-mediated cytotoxicity of tumor cells) that enables methods of treatment regardless of solid tumor type, and whether or not the tumor expresses NECL5. The tumor may be in any organ, for example, kidney, brain, prostate, ovary, breast, lung, colon, and skin. Various types of tumors may be treated, including, for example, glioblastoma, medulloblastomas, carcinoma, adenocarcinoma, etc. Other examples of tumors include, adrenocortical carcinoma, anal cancer, appendix cancer, grade I (anaplastic) astrocytoma, grade II astrocytoma, grade III astrocytoma, grade IV astrocytoma, atypical teratoid/rhabdoid tumor of the central nervous system, basal cell carcinoma, bladder cancer, breast sarcoma, bronchial cancer, bronchoalveolar carcinoma, cervical cancer, colon cancer, colorectal cancer, craniopharyngioma, endometrial cancer, endometrial uterine cancer, ependymoblastoma, ependymoma, esophageal cancer, esthesioneuroblastoma, Ewing's sarcoma, extracranial germ cell tumor, extragonadal germ cell tumor, extrahepatic bile duct cancer, fibrous histiocytoma, gall bladder cancer, gastric cancer, gastrointestinal carcinoid tumor, gastrointestinal stromal tumor, gestational trophoblastic tumor, gestational trophoblastic tumor, glioma, head and neck cancer, hepatocellular cancer, Hilar cholangiocarcinoma, hypopharyngeal cancer, intraocular melanoma, islet cell tumor, Kaposi sarcoma, Langerhans cell histiocytosis, large-cell undifferentiated lung carcinoma, laryngeal cancer, lip cancer, lung adenocarcinoma, malignant fibrous histiocytoma, medulloepithelioma, melanoma, Merkel cell carcinoma, mesothelioma, endocrine neoplasia, nasal cavity cancer, nasopharyngeal cancer, neuroblastoma, oral cancer, oropharyngeal cancer, osteosarcoma, ovarian clear cell carcinoma, ovarian epithelial cancer, ovarian germ cell tumor, pancreatic cancer, papillomatosis, paranasal sinus cancer, parathyroid cancer, penile cancer, pharyngeal cancer, pineal parenchymal tumor, pineoblastoma, pituitary tumor, pleuropulmonary blastoma, renal cell cancer, respiratory tract cancer with chromosome 15 changes, retinoblastoma, rhabdomyosarcoma, salivary gland cancer, small cell lung cancer, small intestine cancer, soft tissue sarcoma, squamous cell carcinoma, squamous non-small cell lung cancer, squamous neck cancer, supratentorial primitive neuroectodermal tumor, supratentorial primitive neuroectodermal tumor, testicular cancer, throat cancer, thymic carcinoma, thymoma, thyroid cancer, cancer of the renal pelvis, urethral cancer, uterine sarcoma, vaginal cancer, vulvar cancer, and Wilms tumor. In one embodiment, the tumor is from the group consisting of a brain tumor, renal cell carcinoma, prostate tumor, bladder tumor, esophageal tumor, stomach tumor, pancreatic tumor, colorectal tumor, liver tumor, gall bladder tumor, breast tumor, lung tumor, head and neck tumor, skin tumor, melanoma, and sarcoma.

In some embodiments, the first tumor and distal tumor may each comprise a solid tumor. For cancer in which multiple occurrences of tumor develop on the same organ (e.g., melanoma and the skin), a distal tumor may comprise a tumor several centimeters (e.g., on the same limb) or more (on a different limb) from a first tumor. The distal tumor may comprise a metastases of the first tumor; a tumor of the same tumor type (e.g., both the first tumor and distal tumor may be a melanoma); or a tumor type different than the first tumor but sharing one or more tumor antigens (“shared tumor antigen”) with the first tumor (e.g., first tumor may be a head and neck tumor, and the distal tumor may be a glioma). Thus the term distal tumor can be used interchangeably with the term metastasis and metastatic tumor. The method may further comprise a reduction in in one or more of rate of tumor growth or proliferation, tumor size, or tumor burden, in the first tumor as a result of treatment with the chimeric poliovirus. The method may further comprise administering a poliovirus vaccine booster (e.g., inactivated polio vaccine trivalent inactivated IPOL from Sanofi-Pasteur), or oral polio vaccine booster) between 6 months and 1 week prior to administering chimeric poliovirus to the first tumor. The method may further comprise administering a therapeutically effective amount of an immune checkpoint inhibitor to the individual either prior to, concurrent with, or subsequent to, treatment of the first tumor with chimeric poliovirus. The method may further comprise multiple administrations of the chimeric poliovirus to the first tumor. The method may further comprise achieving a complete pathologic response in distal tumors.

Optionally, individuals having tumor may be stratified for treatment on the basis of NECL5 (CD155, poliovirus receptor) expression by the individual's tumor prior to treatment according to the methods described herein. This can be assayed at the RNA or protein level, using probes, primers, or antibodies, for example. The NECL5 expression may guide the decision to treat or not treat with the chimeric poliovirus. The NECL5 expression may also be used to guide the aggressiveness of the treatment, including the dose, frequency, and duration of treatments. Antibodies to NECL5 (CD155) are commercially available and may be used. NECL5 RNA expression can also be assayed, using methods known in the art. In one embodiment, the subject to be treated is a subject in which NECL5⁺ distal tumors have been detected. In another embodiment, the subject to be treated is s subject in which NECL5⁺ primary tumors have been detected. In a further embodiment, the subject has NECL5⁺ primary and NECL5⁺ distal tumors detected.

Optionally, following observance of the reduction of distal tumor as a result of treatment by a method of the invention, treatment of the individual may further include treatment by one or more of chemotherapy, biological therapy, and radiotherapy. Such further treatment may be advised by a medical practitioner where such further treatments represent modalities that may be current standard of care for treatment of certain human tumors.

Monitoring one more distal tumors in the individual for a change in tumor properties following contacting tumor cells of the first tumor with the chimeric poliovirus can be by any means known in the art. In one aspect, imaging may be used to monitor distal tumor for such change, using techniques known in the art for imaging tumor. Imaging may comprise visual imaging (e.g., for distal tumor occurring on the skin), positronic emission tomography, magnetic resonance imaging, radiography, computed tomography, ultrasound imaging, and nuclear medicine imaging. Use of such methods of monitoring are within the understanding of one skilled in the art.

Treatment of the individual with any methods of the invention may result in a therapeutic benefit comprising one or more of: improved overall survival; improved disease-free survival; decreased likelihood of recurrence (in the primary organ and/or distant recurrence); decreased incidence of metastatic disease; an increased antitumor immune response; or an improvement in overall objective response rate using the appropriate response assessment criteria known to those skilled in the art and depending on the type of cancer treated (e.g., for lymphoma, see Cheson et al., 2014, J. Clin. Oncology32 (27):3059-3067; for solid nonlymphoid tumors, Response Evaluation Criteria In Solid Tumors (RECIST). The term “treatment” or “treating” further may be characterized by at least one of the following: (a) reduction in one or more properties of the tumor, especially the distal tumors, (b) the reducing, slowing or inhibiting the growth of cancer and cancer cells, including slowing or inhibiting the growth of distal tumor cells; (c) preventing the further growth of distal tumors; (d) reducing the metastasis of cancer cells within a subject; (e) reducing or ameliorating at least one symptom of cancer. The reducing or inhibiting of at least one property of the tumor or distal tumor includes reduction in one or more of rate of tumor growth or proliferation, tumor size, or tumor burden.

In some embodiments, the treatment results in a complete pathologic response. The term “complete pathologic response” refers to the ability of the treatment to reduce the tumor size or burden to undetectable levels.

The present invention further provides a chimeric human poliovirus or a composition comprising a chimeric poliovirus described herein for use in the treatment of distal tumors. The composition may further comprise a pharmaceutically acceptable carrier. As used herein, the term “pharmaceutically acceptable carrier” means any compound or composition or carrier medium useful in any one or more of administration, delivery, storage, stability of an immunotherapeutic agent, treatment agent, composition or combination described herein. These carriers are known in the art to include, but are not limited to, a diluent, water, saline, suitable vehicle (e.g., liposome, microparticle, nanoparticle, emulsion, capsule), buffer, tracking agents, medical parenteral vehicle, excipient, aqueous solution, suspension, solvent, emulsions, detergent, chelating agent, solubilizing agent, salt, colorant, polymer, hydrogel, surfactant, emulsifier, adjuvant, filler, preservative, stabilizer, oil, binder, disintegrant, absorbant, flavor agent, and the like as broadly known in the pharmaceutical art. Suitably, the pharmaceutically acceptable carrier is a carrier that maintains the structure and activity of the chimeric poliovirus before administration.

The chimeric poliovirus or chimeric human poliovirus used herein is a chimeric poliovirus comprising a Sabin type I strain of poliovirus with a human rhinovirus 2 (HRV2) internal ribosome entry site (IRES) in said poliovirus' 5′ untranslated region between said poliovirus' cloverleaf and said poliovirus' open reading frame as described in U.S. Pat. No. 10,398,743, PCT Publication No. WO2016201224 and WO2014081937, and Ochai et al, 2006, Clinical Cancer Research 12(4): 1350-1354. the contents of which are incorporated by reference in its entirely regarding the chimeric poliovirus. The chimeric poliovirus is preferably PVS-RIPO. Briefly, PVS-RIPO was derived from a previous version of the recombinant virus, PV1(RIPO), by substituting the coding region of poliovirus type 1 (Mahoney) with its counterpart from the type 1 live attenuated Sabin [PV1(S)] vaccine strain. The synthesis of PVS-RIPO was reported previously (Ochiai H, Moore SA, Archer GE, et al. Treatment of intracerebral neoplasia and neoplastic meningitis with regional delivery of oncolytic recombinant poliovirus. Clin Cancer Res 2004;10:4831-8.). In brief, infectious PV1(S) cDNA [clone pS1 (T7)], kindly provided by A. Nomoto (University of Tokyo, Japan), was digested with AvaI The resulting 7.0-kb restriction fragment was ligated with a PCR fragment amplified from PV1 (RIPOS) using primers (a) 5V-GGGTCGACTAATACGACTCCTATAGTAAAACAGCTCTGGGGTTGT-3V (SEQ ID NO:1) and (b) 5V-CCATTTCTCGGGCACTGGAGCG-3V (SEQ ID NO:2) and pBS vector DNA (New England Biolabs, Beverly,MA) digested with AvaI and Sall. PVS-RIPO cDNA was processed for rederivation of virus as described before (Gromeier M, Alexander L,Wimmer E. Internal ribosomal entry site substitution eliminates neurovirulence in intergeneric poliovirusr ecombinants. Proc Natl Acad Sci USA 1996; 93:2370-5.).

While the terms used in the description of the invention are believed to be well understood by one of ordinary skill in oncology and medicine, definitions, where provided herein, are set forth to facilitate description of the invention, and to provide illustrative examples for use of the terms.

As used herein, the terms “a”, “an”, and “the” mean “one or more”, unless the singular is expressly specified (e.g., singular is expressly specified, for example, in the phrase “a single agent”).

The terms “first”, “second”, and “additional”, are used herein for purposes of distinguishing between two tumors, or between two compounds, or between two or more compositions, or between two or more steps of a method, as will be clearer from the description.

“Maintenance therapy” is used herein to refer to therapeutic regimen that is given to reduce the likelihood of disease progression or recurrence. Maintenance therapy can be provided for any length of time depending on assessment of clinical parameters for assessing response to therapy.

“Shared tumor antigens” is used herein to refer to tumor-associated antigens that are expressed by or on more than one tumor type. For example, cancer-testis antigens are shared tumor antigens that are expressed in histologically different human tumor tissues (e.g., melanoma; breast, bladder, colon, and lung carcinomas), including BAGE, MAGE, GAGE, NY-ESO-1, and SSX. Differentiation antigens are shared tumor antigens expressed by melanomas and tumors of epithelial origin (e.g., prostate, colon, and breast carcinomas), including CEA, PSA, Tyrosinase, GP100, Mammaglobin-A, and Mart-1/Melan-A. Overexpressed tumor-associated antigens are shared tumor antigens that have been detected in different types of tumors (e.g., esophagus, liver, breast, colon, pancreas, ovary, bladder and prostate carcinomas) as well as in many normal tissues, including p63, Her-2/neu, livin, survivin, and MUC-1.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of those elements”. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

All publications, patent applications, patents, and other references mentioned herein are incorporated by reference in their entirety. In the case of conflict, the present specification, including definitions, will control.

The above disclosure generally describes aspects of the invention. A more complete understanding can be obtained by reference to the following specific examples, which are provided herein for purposes of illustration only, and are not intended to limit the scope of the range of techniques and protocols in which the compositions and methods of the present invention may find utility, as will be appreciated by one of skill in the art and can be readily implemented. The present invention has been described in terms of one or more preferred embodiments, and it should be appreciated that many equivalents, alternatives, variations, and modifications, aside from those expressly stated, are possible and within the scope of the invention.

### EXAMPLE 1

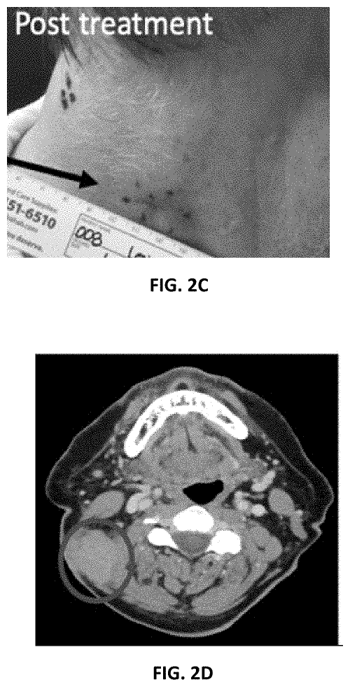

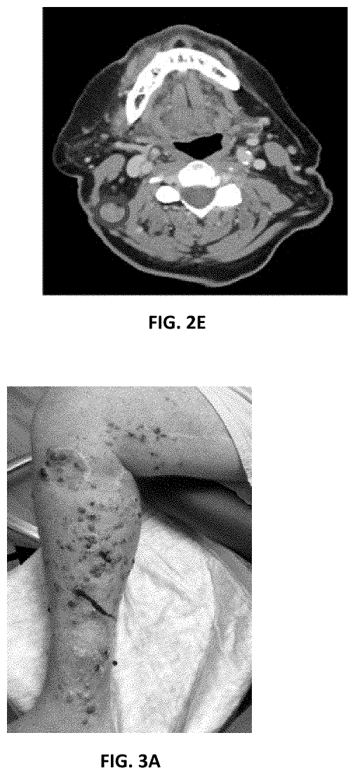

Provided is an illustrative example of a method of treatment wherein treatment the first tumor in the individual results in a reduction of one or more tumor properties of the distal tumors. In this example, the patients were treated with chimeric poliovirus in a Phase I study. The inclusion criteria include that the patient received a booster immunization with a poliovirus vaccine at least 1 week prior to being treated with the chimeric poliovirus, and the patient must have failed standard of care treatment for that tumor type. Patient A was diagnosed in March 2019 as having melanoma (biopsy confirmed, BRAF wild type). Patient A was then treated with 3 cycles of nivolumab (anti-PD-1 check point inhibitor antibody), then 3 cycles of pembrolizumab (anti-PD-1 checkpoint inhibitor antibody). Patient A′s tumors progressed despite receiving treatment with immune checkpoint inhibitors and, thus, failed checkpoint inhibitor therapy. Patient A′s last dose of anti-PD-1 antibody was 1 month prior to first PVSRIPO injection. Patient A received a poliovirus vaccine booster at least a week before the first PVSRIPO injection. At this point, patient A had melanoma that comprised 4 palpable neck nodules which were also scanned using computed tomography (CT). Patient A was administered PVSRIPO (titer of 1×10⁸) into a first melanoma tumor (See FIG. 2A, tumor marked “1”). The 4 tumors were then measured for tumor size at day 21 following administration of PVSRIPO to the first treated tumor. As shown in Table 1 and in FIG. 2B, there was a reduction in measured tumor size of a tumor (tumor “2”) adjacent to the first treated tumor after 21 days. Tumor “2” was then injected with PVSRIPO (titer of 1×10⁸; day 21 after the first tumor was treated). At day 42 (21 days following the treatment of the second tumor), the 4 tumors were then measured for tumor size. As shown in Table 1 and FIG. 2C, in addition to a reduction in size of the treated tumors, there was a measurable reduction in size in distal tumors not receiving PVSRIPO treatment.

TABLE 1

| | | Tumor size | Tumor size |
|---|---|---|---|
| | | 21 days | 42 days |
| | Tumor size | after PVSRIPO | (treatment of |
| | prior to | treatment of | a first tumor and |
| Tumor | treatment | first tumor | a second tumor) |
| tumor 1 | 3 × 3 mm | 2.5 × 2.5 mm | 1.2 × 1.5 mm |
| tumor 2 | 2.5 × 3 mm | 2.5 × 2.1 mm | 1.5 × 1.5 mm |
| tumor 3 | 1 × 1 mm | 1 × 1 mm | 0.8 × 0.5 mm |
| tumor 4 | 1 × 1 mm | 1 × 1 mm | 0.5 × 0.5 mm |

To obtain an objective response relative to treatment with the chimeric poliovirus, Patient A was followed at least 60 days after treatment was initiated with PVSRIPO and CT scans of the tumor were compared form before and after treatment with PVSRIPO. As shown when comparing FIG. 2D (CT scan of tumor before treatment with chimeric poliovirus, denoted by circle) with FIG. 2E (CT scan of tumor 63 days after treatment with chimeric poliovirus), the largest node containing tumor was decreased by about 75% in size, and there was also a decrease in size noted for nodes containing tumor adjacent to the site of treatment.

### EXAMPLE 2

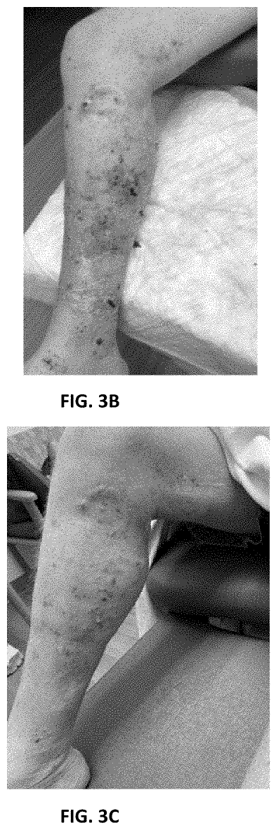

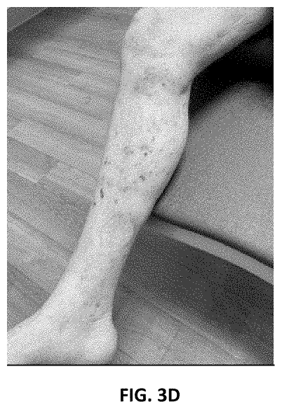

Provided is another illustrative example of a method of treatment wherein treatment the first tumor in the individual results in a reduction of one or more tumor properties of distal tumors of the individual. Patient B had an initial diagnosis of melanoma in May 2018, and a second primary melanoma on the ipsilateral in extremity diagnosed in April 2019. Patient B underwent excision and sentinel node of both lesions (BRAF wild type), and both sentinel nodes were negative. Patient B recurred in July 2019 with multiple in-transit lesions on an extremity (right leg). Patient B was treated with 4 cycles of nivolumab. Patient B′s tumors progressed despite receiving treatment with immune checkpoint inhibitor and, thus, failed checkpoint inhibitor therapy. Patient B′s last dose of anti-PD-1 antibody was 15 days prior to first PVSRIPO injection. Patient B received a poliovirus vaccine booster at least a week before the first PVSRIPO injection. At this point, patient B had numerous (>50 lesions) on the right leg (see FIG. 3A), but no other metastatic disease was observed from CT scans. Patient B was administered PVSRIPO (titer of 1×10⁸) into a first melanoma tumor. Five tumors were selected for measurement and followed using accepted guidelines for response criteria modified for cutaneous disease (iRECIST guidelines). FIG. 3B shows the response of all tumors (treated tumor and distal tumor) 9 days after treatment of the first tumor with PVSRIPO. As shown in Table 2, there was a reduction in measured tumor size of at least one tumor distal to the first treated tumor after 21 days. A second tumor was then injected with PVSRIPO (titer of 1×10⁸; day 21 after the first tumor was treated). At day 42 (21 days following the treatment of the second tumor), tumor size was then measured. As shown in Table 2 and FIG. 3C, there was a measurable reduction in size in distal tumors not receiving PVSRIPO treatment.

TABLE 2

| | | Tumor size | Tumor size |
|---|---|---|---|
| | | 21 days | 42 days |
| | Tumor size | after PVSRIPO | (treatment of |
| | prior to | treatment of | a first tumor and |
| Tumor | treatment | first tumor | a second tumor) |
| tumor 1 | 1.5 × 1 mm | treated, resected | resected |
| tumor 2 | 1 × 1 mm | 0.8 × 0.8 mm, | 0.8 × 0.6 mm |
| | | then treated | |
| tumor 3 | 1 × 0.8 mm | 1 × 0.5 mm | 0.8 × 0.4 mm |
| tumor 4 | 2 × 1.5 mm | 1.5 × 1.3 mm | 0.6 × 0.5 mm |
| tumor 5 | 1 × 0.5 mm | 0.8 × 0.8 mm | 0 |

To obtain an objective response relative to treatment with the chimeric poliovirus, Patient B was followed at least 90 days after treatment was initiated with PVSRIPO. Biopsy of the lesion 1 that was treated with chimeric poliovirus and a distal lesion (untreated) on day 53 post-injection of the treated lesion showed complete pathologic response in both lesions (no evidence of tumor). Restaging CT scans performed 2 months after treating tumor lesion with a first injection of chimeric poliovirus PVSRIPO showed no evidence of other metastatic disease. As shown in FIG. 3D, as compared to FIG. 3A, after 90 days posttreatment of 3 tumor lesions, an abscopal effect is clearly evident as the distal lesions (untreated with chimeric poliovirus) appeared completely resolved. Some pigmentation remained but biopsy of pigmentation showed macrophages only, and no viable tumor cells, thereby confirming a complete pathologic response (e.g., observed absence of viable tumor). At this time, Patient B shows no evidence of malignant disease.

1. A method of treating one or more distal tumors in an individual having a first tumor, the method comprising:
contacting tumor cells of the first tumor with a chimeric poliovirus comprising a Sabin type I strain of poliovirus with a human rhinovirus 2 (HRV2) internal ribosome entry site (IRES) in said poliovirus' 5′ untranslated region between said poliovirus' cloverleaf and said poliovirus' open reading frame forming a first tumor treated with the chimeric poliovirus, and
wherein the one or more distal tumors are not contacted with the chimeric poliovirus;
wherein treatment of the first tumor in the individual results in a reduction of one or more tumor properties of one or more of the distal tumors.

2. The method of claim 1, further comprising after the contacting step:
monitoring the one more distal tumors in the individual for a change in tumor properties following contacting tumor cells of the first tumor with the chimeric poliovirus

3. The method of claim 1, wherein the reduction of one or more tumor properties of the one or more distal tumors comprises a reduction in one or more of rate of tumor growth or proliferation, tumor size, or tumor burden.

4. The method of claim 1, wherein tumor is solid tumor.

5. The method of claim 1, wherein distal tumor comprises a metastases.

6. The method of claim 1, wherein the first tumor and distal tumor both express a shared tumor antigen.

7. The method of claim 1, wherein the contacting comprises administering a composition comprising a chimeric poliovirus to a subject.

8. (canceled)

9. The method of claim 1, further comprising administering a poliovirus vaccine booster prior to administering the chimeric poliovirus to the first tumor.

10. The method of claim 1, further comprising treating the individual with anti-cancer therapy selected from the group consisting of immunotherapy with an immune checkpoint inhibitor, radiation therapy, or chemotherapy subsequent to a reduction of one or more tumor properties of the one or more distal tumors.

11. (canceled)

12. The method of claim 1, wherein the tumor is selected from the group consisting of a brain tumor, renal cell carcinoma, prostate tumor, bladder tumor, esophageal tumor, stomach tumor, pancreatic tumor, colorectal tumor, liver tumor, gall bladder tumor, breast tumor, lung tumor, head and neck tumor, skin tumor, melanoma, and sarcoma.

13. The method of claim 1, wherein the distal tumor expresses NECL5 (nectin- like protein 5).

14. The method of claim 1, wherein prior to administering the chimeric poliovirus to the individual, the method comprises the step of detecting expression of NECL5 in the individual's distal or primary tumor.

15. The method of claim 14, wherein the method comprises the step of detecting expression on NECL5 in the subject's distal tumor.

16. The method of claim 10, wherein the immune checkpoint inhibitor is selected from the group consisting of an anti-PD-1 antibody, an anti-PDL-1 antibody, an anti-CTLA4 antibody and fragments thereof.

17. The method of claim 16, wherein the immune checkpoint inhibitor further comprises a pharmaceutically acceptable carrier.

18. The method of claim 1, wherein treatment results in a complete pathologic response.

19. The method of claim 1, wherein the chimeric poliovirus is PVSRIPO.

20. A chimeric poliovirus comprising a Sabin type I strain of poliovirus with a human rhinovirus 2 (HRV2) internal ribosome entry site (IRES) in said poliovirus' 5′ untranslated region between said poliovirus' cloverleaf and said poliovirus' open reading frame, for use in treating one or more distal tumors in an individual having a first tumor, optionally which expresses NECL5, wherein the chimeric poliovirus is administered to the first tumor.

21. The chimeric poliovirus of claim 20, further comprising a pharmaceutically acceptable carrier.

22. The chimeric poliovirus of claim 20, wherein the chimeric poliovirus is PVSRIPO.
